(19) United States
(12) Patent Application Publication
(10) Pub. No.: US 2007/0190642 A1
(43) Pub. Date: Aug. 16, 2007

(54) Concentrators for Luminescent Emission

(75) Inventor: Steven J. Boege, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/675,546

(22) Filed: Feb. 15, 2007

Related U.S. Application Data
(60) Provisional application No. 60/774,462, filed on Feb. 16, 2006.

(51) Int. Cl. C12M 1/34; G01N 21/64

(52) U.S. Cl. 435/288.7; 422/82.08

(57) ABSTRACT

System and methods for concentrating collected flux in the form of excitation light or luminescence. The collected flux may be directed, propagated, and concentrated in various manners and may be localized into a generally small area. Concentrators may be advantageously implemented in flexible and efficient manners relative to conventional configurations where a single lens acts alone to direct the light to a detector.

### CROSS-REFERENCE TO RELATED APPLICATIONS

This application claims priority to U.S. provisional Application No. 60/774462, filed Feb. 16, 2006 entitled “Concentrators for Luminescent Emission”. Additionally, this application is related to commonly assigned U.S. application Ser. No. 11/356,497, filed Feb. 16, 2006 (based upon U.S. Provisional Application No. 60/653,606 filed Feb. 16, 2005), and entitled “Axial Illuminator for Capillary Electrophoresis” the disclosures of which are herein incorporated by reference in their entirety.

### FIELD

The present teachings relate to devices and methods for generating and detecting luminescence.

### INTRODUCTION

Molecular biology and other sciences frequently utilize luminescent detection because of its wide acceptance and sensitivity. Examples of methods utilizing luminescent detection include chromatography and electrophoresis. Luminescent light can be generated by exciting dyes or labels in a sample using excitation light or by chemical means which result in the production of luminescent emissions. The luminescent light emitted can be isotropic or diffuse due to low concentrations of dye or luminescent label in the sample. In many instances, it is desirable to collect more of the radiated light from a sample to increase the efficiency of luminescent detection.

The luminescent light emitted from a labeled sample can be proportional to the amount of excitation light that can be directed to the detection zone. For non-coherent light sources, such as, for example, light emitting diodes (LEDs), filament lamps, and arc lamps, only a fractional amount of the light is typically directed towards the sample container. Additionally luminescence from samples may be diffuse and it may be desirable to concentrate these emissions towards the detection zone.

Luminescent light detection systems can additionally benefit from smaller, lower cost, and lower power excitation light sources being either coherent or non-coherent in origin. Additionally, it may be desirable to provide mechanisms by which to propagate excitation light energy to a detection zone by, for example, coupling illumination to propagate at least a portion of luminescence associated with the sample container to the detection zone.

### SUMMARY

It is to be understood that both the foregoing general description and the following description of various embodiments are exemplary and explanatory only and are not restrictive. In various embodiments, the present teachings describe a system for illuminating a biological sample having a responsive luminescent label, the system comprising: an illumination source that emits energy of a type capable of a generating a response in the luminescent label; a concentrator that receives at least a portion of the energy emitted by the illumination source and concentrates the received energy as the energy propagates through said concentrator and wherein the concentrator is configured to emit said concentrated energy with characteristic properties; and a sample container containing the biological sample and adapted to receive the concentrated energy emitted from the concentrator wherein the concentrated energy effectuates a detectable luminescent emission arising from the label of the biological sample.

In other embodiments, the present teachings describe a system for detecting luminescent emissions arising from a label associated with a biological sample, the system further comprising: a sample container containing the biological sample from which luminescent emissions arising from the label are emitted; a concentrator that receives at least a portion of the luminescent emissions arising from the label and concentrates the luminescent emissions they are propagated through said concentrator and wherein the concentrator is configured to emit said concentrated luminescent emissions with characteristic properties; and a detector that receives the concentrated luminescent emissions and generates a signal in response to the received concentrated luminescent emissions.

### BRIEF DESCRIPTION OF THE DRAWINGS

The accompanying drawings, which are incorporated in and constitute a part of this specification, illustrate various embodiments. In the drawings,

FIG. 1 illustrates an exemplary a concentrator that may be devised for use with a detector to capture light emitted from a sample container.

FIG. 2 illustrates an exemplary concentrator that operates to concentrate excitation light emitted from a light source.

FIGS. 3A-D illustrate exemplary concentrators that comprise a frustum of a cone or other elongate structure that concentrates the light.

FIGS. 4A-B illustrate exemplary concentrators comprising a compound parabolic concentrator that concentrates the light.

FIGS. 5A-C illustrate exemplary concentrators having properties of total internal reflection shown in side view.

FIGS. 6A-D illustrate exemplary dielectric compound parabolic and wedge concentrators.

### DETAILED DESCRIPTION OF VARIOUS EMBODIMENTS

Reference will now be made to various exemplary embodiments, examples of which are illustrated in the accompanying drawings. Wherever possible, the same reference numbers are used in the drawings and the description to refer to the same or like parts.

The term “light source” as used herein refers to a source of irradiance (can be measured in photons/mm²) that can provide illumination light or excitation energy that results in luminescent emission from a labeled sample. In various aspects, irradiance may be related to luminescent emissions. For example, fluorescent light from a label is generally proportional to the number of photons available from the light source for excitation. Light sources can include, but are not limited to, lasers, solid state laser, laser diode, diode solid state lasers (DSSL), vertical-cavity surface-emitting lasers (VCSEL), LEDs, phosphor coated LEDs, organic LEDs, inorganic-organic LEDs, LEDs using quantum dot technology, LED arrays, filament lamps, arc lamps, gas lamps, and fluorescent tubes. Light sources can have high irradiance, such as lasers, or low irradiance, such as LEDs.

The term “non-coherent light” as used herein refers to irradiance from a non-laser light source. Non-coherent light sources can include, but are not limited to LEDs, phosphor coated LEDs, organic LEDs, inorganic-organic LEDs, LEDs using quantum dot technology, LED arrays, filament lamps, arc lamps, gas lamps, and fluorescent tubes.

The term “luminescent light”, “luminescent energy”, “emission light”, “emission energy” and “luminescence” as used herein refers to light or energy emitted by a sample. Such light or energy may arise in response to excitation light or energy provided by the light source or may arise from a chemical property or characteristic of the label itself as may be the case for a chemiluminescent label or sample.

Luminescent light can be emitted in all directions. Luminescent light can be related to detection signal with the detection signal being generally proportional to the number of photons or quanta of luminescent light or energy collected from the sample. Luminescent light can be emitted by a sample excited by excitation light, as in fluorescence, or electrically excited.

The term “concentrator” as used herein refers to a singlet or assembly of components that can focus, direct, and/or moderate excitation or emission light (e.g. light arising from the light source and/or light arising from the label associated with the sample). The concentrator may be adapted to receive light (excitation or emission light), propagate the light within the concentrator, and allow the light to pass from a portion (e.g. end) of the concentrator with desired or selected characteristic properties. These characteristic properties may include by way of example selecting for a particular wavelength of light, selecting for a desired dimensionality of light exiting the concentrator (for example, beam size or diameter), selecting for a particular intensity of light exiting the concentrator, selected for a particular coherence of the light exiting the concentrator, reducing cross-talk between sample containers, selectively illuminating sample containers, selectively detecting luminescence from selected sample containers, etc. The shape and size of the concentrator may be adapted to suit a particular application or group of applications and may be shaped in a manner so as to confer the desired characteristic properties upon the light entering the concentrator. Possible suitable shapes for the concentrator that may be used in various contexts include a cone, a truncated sphere, a hyperhemisphere, a spherical surface combined with a cylindrical surface, a spherical surface combined with a planar surface, etc. The components of a concentrator can be bonded or coupled with compositions such as a solid or a fluid of suitable refractive index that does not substantially interfere with the desired characteristic properties of the excitation or emission light.

The refractive index or selected optical properties of the concentrator can be similar to the refractive index or selected optical properties of a material which abuts, joins, contacts, or is in proximity with the concentrator. In one embodiment, a coupling element may be used in connection with the concentrator where the coupling element facilitates transmission of the light into or out of the concentrator preserving selected optical properties or characteristics of the light. Additionally, the coupling element can be less than or equal to the index of refraction of the concentrator or may not be used for a particular application. According to various embodiments, the index of refraction of the coupling element can be from about 1.43 to about the index of refraction of the concentrator. The concentrator can be constructed of BK7, PBH71, LaSFN9, or other high index glasses, plastics, such as, for example, methyl methacrylate, polycarbonate, or a combination of glass and plastic. The concentrator can be “doped”, “embedded”, “coated” with various materials to confer desired optical properties to the concentrator. For example, the concentrator may be configured to propagate light within the concentrator through total internal reflection (TIR) through the use of selected surface coatings. Additionally, the concentrator may be configured as a hollow structure, solid structure, fluid-filled structure, gel-matrix containing structure or other suitable configurations to convey the desired optical properties to the concentrator.

The term “optical component” as used herein can refer to lenses, gratings, mirrors, filters or other optical components that are used in connection with the concentrator to direct, shape, focus, align, condition, and/or manipulate excitation or emission light. A system that utilizes the concentrator can include multiple optical components and may be configured in numerous possible manners to achieve desired propagation of light within the system.

The term “detector” as used herein refers to any component or system of components that can detect light or energy including a charged coupled device (CCD), back-side thinned CCD, cooled CCD, a photodiode, a photodiode array, a photo-multiplier tube (PMT), a PMT array, complimentary metal-oxide semiconductor (CMOS) sensors, CMOS arrays, a charge-injection device (CID), CID arrays, etc.

The term “sample container” as used herein refers to any structure that provides containment or support to the sample. The sample container can be transparent or optically permissive to provide entry or passage of excitation light and exit or passage of luminescent light. The sample container can be constructed of glass, plastic such as low fluorescence plastic, fused silica such as synthetic fused silica or synthetic quartz, etc. The sample container can take any shape including one or more tubes (various types), capillaries, assemblies of capillaries, etched channel plates, molded channel plates, embossed channel plates, wells in a multi-well tray, chambers in a microcard, regions in a microslide, cuvettes, microcards, etc.

The term “label” as used herein refers to any label in any form that may be used to generate luminescent emissions. Labels may include by way of example fluorescing dyes, luminophores, upconverting phosphors and quantum dots that may generate luminescent emissions. In one exemplary application, a fluorescing dye can emit fluorescent light via fluorescence resulting from exposure to excitation energy of a suitable wavelength or range of wavelengths. Fluorescent dyes can be used to emit different colored light depending on the dyes used, similarly other labels can be used to generate light or energy of different wavelengths and may be used in combination with one another. Several labels will be apparent to one skilled in the art of label chemistry as related to labeling and detection of biological molecules. One or more wavelengths of luminescent emissions can be collected for each label to provide identification of the label or labels detected. The label can be based or associated with other chemical species and may be linked to the biological sample of interest including by way of example proteins, carbohydrates, nucleic acids, etc.

The term “cross-talk” as used herein refers to luminescence emitted from one sample or sample container appearing in, interfering with, or interacting with the detection position of another sample. The samples can be in different sample containers or in the same sample containers. The cross-talk can be the result of reflection, scattering, and/or refraction from components in the system.

The present teachings relate to apparatus and methods for exciting and/or collecting luminescence. Turning to collection of luminescence, the sample can include a label in a fluid or solid. The sample emits luminescent light in all directions. The collection system including the detector collects a portion of this light.

### Exemplary Applications for Concentrators

In certain applications, detector architectures comprising single element photodetectors such as photo-multiplier tubes (PMTs) or photodiodes can be used in instrument platforms as well as for research purposes. In these systems, lenses can be used to image emitters or pupils of collection optics onto photodetectors. In such cases, a certain irradiance and a certain power are realized on the active planar area of the photodetector. In one aspect, the noise characteristics of the photodetectors may be evaluated as proportional to their area. Consequently, concentrating a collected illumination flux into a smaller area provides a mechanism by which to allow use of less noisy, less costly photodetectors that can be more rapidly/readily interrogated.

In one aspect of the present teachings, an optic component (e.g. lens, mirror, etc.) capable of forming an image on a photodetector can be replaced/substituted with a concentrator capable of transmitting sample luminescence on a photodetector. In various embodiments, such irradiance may be greater that that produced by the comparable image-forming lens. Additionally, the concentrator may be used to transmit excitation light to a sample container replacing one or more optic components that might otherwise be used. The concentrator may further impart desired characteristic properties to the excitation or emission light to improve luminescent detection of sample labels.

Various configurations of non-imaging optics may be defined generally as concentrators. In various embodiments, the concentrator system may optionally comprise a lens or other optical components. In various embodiments, a concentrator may have a hollowed or solid structural configuration. Concentrator systems comprising solid concentrator elements may include various configurations, such as, a lens surface integral to the solid concentrator, a discrete (extrinsic) lens element non-integral to the solid concentrator, or may be configured as a concentrator system without lenses.

Concentrator systems including those lacking lenses may comprise at least one reflective surface or a surface having properties of total internal reflectance (TIR). TIR properties may further result from coated or uncoated surfaces having appropriate optical properties. The reflective surface may be rotated about an axis that is substantially normal to and through the center of the face of the photodetector. The reflective surface profile (the distance of the reflector surface from the axis of symmetry measured along an axis normal to the axis of symmetry) can further be a function of distance from the detector. The function can further be configured as a combination of sections of one or more of geometries including for example, linear (cone), spherical, conic (parabola, ellipse, hyperbola), polynomial, or other functions. In one aspect, the axis of symmetry may be anti-parallel to the axis of the function when the function is a conic.

In various embodiments, solid concentrators can optionally use properties of total internal reflection (TIR) to confine/propagate the light. In various embodiments, the solid concentrators can optionally use reflective surface coatings to confine/propagate the light. In various embodiments, the solid concentrators can optionally use reflective surface coatings for selected areas and TIR in other areas to confine/propagate the light. In various embodiments, the solid concentrator can optionally use one or more layers of refractive substantially nonabsorbing materials to effect TIR.

The forgoing configurations desirably provide mechanisms by which collected flux in the form of excitation light or luminescence may be directed, propagated, and/or concentrated. In one aspect, collected flux may be concentrated into a generally small area. Embodiments of the present teachings provide mechanisms by which to use less noisy, less costly, and/or faster responding photodetectors. Such configurations may advantageously implemented in more flexible/efficient manners than other configurations where a single lens acts alone to direct the light to a photodetector.

Trapping, directing, or propagating the light in a concentrator may additionally reduce stray light in the system, which may decrease crosstalk between spectral channels. It will be appreciated that such improvements as described herein may reduce cost and/or improve sensitivity of single element photodetector systems and scanners. Such systems may be particularly desirable in biological applications including detection on fluorescent, chemiluminescent, phosphorescent, or other labels or dyes that may be used for purposes of detecting biological molecules including for example proteins, carbohydrates, and nucleic acids (e.g. RNA, DNA, etc.).

### Exemplary Concentrators for Excitation or Luminescent Emission

Imaging condensers may be used for coupling excitation light from an extended/remote source such as a light emitting diode (LED) source into a sample container such as a cylindrical capillary. Imaging optics regulating the light from an LED may not produce the same irradiance in a sample container such as a capillary as can a suitably focused laser. It certain instances, adequate luminescence can be generated through excitation energy provided using LED light coupled to a capillary with imaging or nonimaging optics.

In various embodiments, the present teachings provide mechanisms to efficiently couple light from an excitation light source into one or more samples. Applications of the present teachings may further be used for directing/propagating light in sample containers. In various embodiments, the present teachings further provide mechanisms for efficient coupling of luminescent emissions out of samples and provide means for improving detection of the luminescent emissions.

As shown in the Figures, a concentrator having a profile as exemplified may be devised. In various embodiments, the concentrator may be extruded along a substantially straight line that is substantially orthogonal to the plane of the concentrator cross section. Such a configuration may be observed as forming a “trough” capable of directing the incident light onto a desired point or area in the concentrator profile. In various embodiments, such direction may be substantially parallel to the direction of extrusion. In various embodiments, flux in the region of the trough may be concentrated to desirably increase the effective illumination of a sample in the region or increase the effect luminescent output for a sample. Such configurations may be used to couple excitation light into a sample container or couple luminescent emission from the sample container to desirably improve the optical characteristics of the system. In various embodiments, concentrator configurations may be used to couple excitation light to, or emission light from, samples.

As shown in the Figures, the concentrator may be formed as a substantially parabolic conic arc. In various embodiments, incident ray bundles may be substantially collimated with the sections being generally rotated versions of substantially parabolic conic arcs. Such configurations as described herein may be used in the concentration of a substantially collimated light for example arising from a laser; an LED, or a lamp light. In various embodiments, as the properties of the incident light, or the desired properties of the exiting light depart from collimated, the cross section may take on other shapes. In various embodiments, the shape or profile of the concentrator may take into account occurrences where input wavefronts depart from substantially planar or spherical dimensionalities (e.g. as aberrations on the incident beams increase). In various embodiments, where the source geometry and condenser prescription are known, the concentrator profile can be tuned to correct for aberrations.

In various embodiments, such as those associated with luminescence applications, the concentrator geometry may be configured such that excitation light/luminescent emissions are substantially trapped/contained in a solid concentrator by total internal reflection TIR. The effects of TIR may be generated by parts of the concentrator surface. Additionally, the effects of TIR may be frustrated with a longpass coating in a sub region through which collection optics may view/detect luminescence in a selected sample region. In various embodiments, truncate hollow or solid troughs may be configured to strategically sacrifice a portion of the concentrators' profile thereby permitting some solid angle for detection.

It will be appreciated that the concentrator configurations of the present teachings provide mechanisms to increase effective irradiance by increasing/shaping beam divergence. In one aspect, the concentrator configurations may be used to produce improved/more efficient irradiances. In certain instances, the concentrator may produce irradiances approaching or substantially equivalent to higher numerical aperture (NA) image forming condenser systems. The concentrators of the present teachings further provide designs that are less expensive to implement and utilize less complicated elements.

The concentrator configurations of the present teachings can be solid or hollow. Additionally, three dimensional (3D) concentrators may be devised as having a generally 2D section rotated about an axis of symmetry. In various embodiments, 3D concentrators can be used to launch, drive, propagate, and/or direct axial illumination into a sample region such as one or more capillaries, wells, or cuvettes. Concentrators can further be devised so that the maximum exit angle is less than 90 degrees. In such instances, the concentrator can be designed with exit angles generally matched to fiber of capillary numerical apertures. In various embodiments, hollow or solid concentrators can be used to couple light into the end face of a sample container capillary or fiber. In such systems, optical contact between the concentrator and the sample container may be conditioned with a selected component such as hardened cement, compliant gelatinous slab, index matching compound, index matching gel, or diffracting compound Additionally, solid concentrators can be used to couple light through the wall of a capillary or fiber.

In various embodiments, a lens on a solid, straight-walled cone may be provided as a compound hyperbolic concentrator. The concentrators of the present teachings may further be used to couple excitation energy or light into sample containers such as capillaries or wells. In various embodiments, the wall profiles of the concentrator may be optimized for regulating or moderating light from the lens to the exit aperture. In certain instances such configurations permit higher concentration ratios of illumination to be devised as compared to the light source illuminating the sample container without the benefit of the concentrator. Furthermore, irradiance can be improved at the exit of the concentrator to provide a desired amount of illumination or luminescent intensity (e.g. maximal), or a wall profile may be selected that results in energy/light leaving the aperture in a substantially parallel manner or at a specified angle. Such configurations may be used for example in sample containers such as capillaries to address angles below which the energy/light is “trapped” or substantially contained within the capillary and does not substantially enter the lumen of the capillary and above which energy/light is not trapped.

In various embodiments, the concentrators may be configured in a “concentration mode” where there are substantially no subsidiary conditions other than TIR imposed. The concentration mode may further yield an optimized or near maximal achievable concentration of light with TIR. In other embodiments, the concentrators may be configured in a “phase-conserving mode” where the exiting rays are generally aligned in a substantially parallel manner forming a desired wavefront.

In various embodiments, solid compound parabolic concentrators may be devised. Such configurations may be used to couple energy/light from an infinite conjugate in through sample container walls. In another application, a concentrator may be used to heat a desired sample region (for example a portion of a sample container, tube, well or capillary) through placement of an absorber location using a concentrator and directing for example an infrared (IR) source into the concentrator.

The aforementioned teachings provide for higher efficiency designs relative to various conventional methods and may be used numerous applications. In one application, a 2D concentrator may comprise a hollow or solid structure for regulating excitation light to and/or luminescent emissions from one or more regions/samples with a selected geometry. In another application, a 3D concentrator may comprise a hollow or solid structure for regulating excitation light to and/or luminescent emissions from end faces of one or more regions/samples with a selected geometry. In still another application, solid 3D concentrators may be devised for regulating excitation light to and/or luminescent emissions through walls of sample containers with selected geometry.

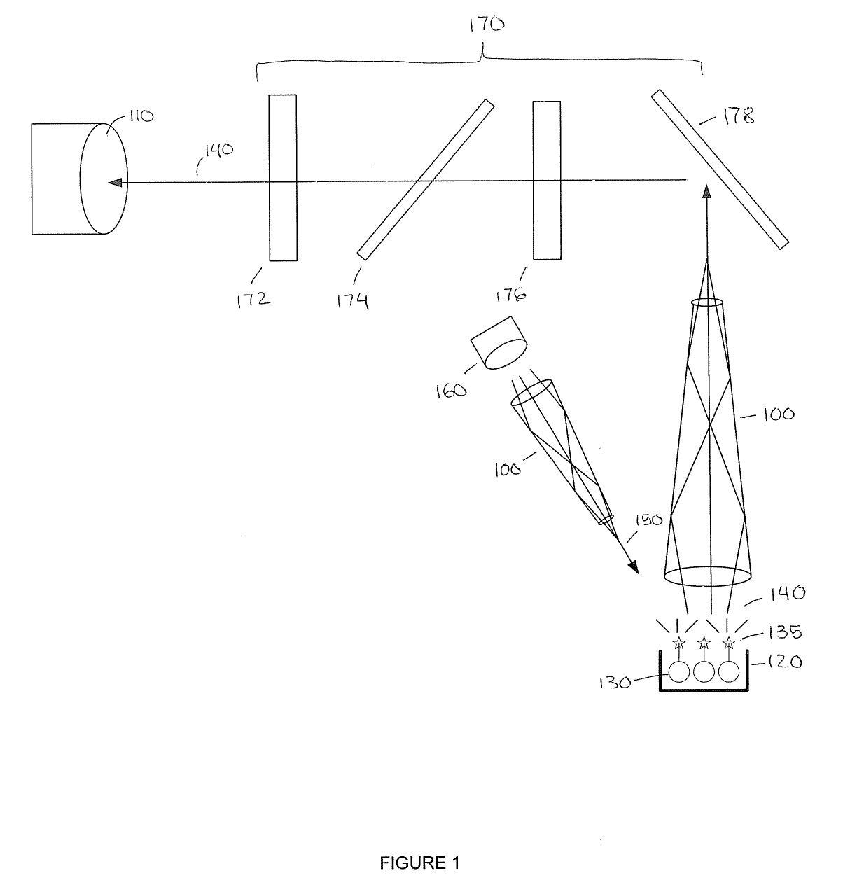

With reference to FIG. 1 a concentrator 100 may be devised for use with a detector 110 such as a photodiode to capture/intercept luminescent energy/light 140 emitted from a sample container 120. As previously described, the sample container 120, may comprises various configurations in which a luminescent sample resides 130. The luminescent sample 130 may generate detectable emissions 140 through auto-illuminating processes such as chemiluminescence or may generate detectable emissions 140 in response to excitation light 150 emitted by a light source 160. As previously indicated, the light source 160 may comprise any of a number of different types of light/energy producing sources which emit excitation light 150 that results in the luminescent emissions 140 arising from labels 135 associated with the sample 130. In various embodiments, the light source 160 may further be coupled with a concentrator 100 as will be described in greater detail hereinbelow. The concentrator 100 for concentrating the excitation light 150 from the light source 160 may be the same concentrator 100 as that used for concentrating the luminescent emissions 140 or alternatively separate concentrators 100 may be used. Alternatively, a system may comprise a single concentrator 100 that concentrates excitation light 150 without any concentrator 100 associated with the concentration of luminescent light 140. Or alternatively, a system may comprise a single concentrator 100 that concentrates luminescent light 140 without any concentrator 100 associated with the concentration of excitation light 150.

In the concentration of excitation light 150 or luminescent light 140 or combinations thereof, various optical components 170 may be used to direct, shape, focus, align, condition, and/or manipulate the excitation light 150 or emission light 140 in desired manners. These optical components 170 may comprise among other things lenses 172, gratings 174, filters 176, mirrors 178, or other optical components that are used in connection with the concentrator 100 to direct/manipulate the excitation light 150 and/or luminescent emissions 140 as desired.

It will be appreciated by one of skill in the art that numerous optical components and optical configurations may be used to achieve various modes and approaches to illuminating sample containers 120 and collecting luminescent emissions 140 for the sample containers 120. Furthermore, the light paths for the excitation light 150 and luminescent emissions 140 need not follow the same optical path and may utilize discrete optical paths. A particular optical path may include some, any or none of the aforementioned optical components 170 used in connection with the concentrator(s) 100 to achieve a desired result in terms of illumination or collection of light.

The concentrator 100 may be used in connection with one or more lenses 172 that may aid in focusing and directing the light energy 140, 150 in a desired manner. Additionally, other optical components 170 such as mirrors 178 and/or gratings 174 may be used along the optical train to focus, direct, or redirect the light onto or away from the concentrator 100. In various configurations, the concentrator 100 desirably allows a smaller detector 110 (for example a photodiode) to be utilized to efficiently capture comparable amounts of luminescent emissions 140 as a larger detector 110 without the benefit of a concentrator 100. In various configurations, the use of the concentrator 100 desirably reduces the cost and/or complexity of the system by allowing less expensive detectors 110 to be used as well as potentially reducing/eliminating the requirement for various optical components 170 along the optical train while retaining comparable or improved detection sensitivity of the luminescent emissions 140.

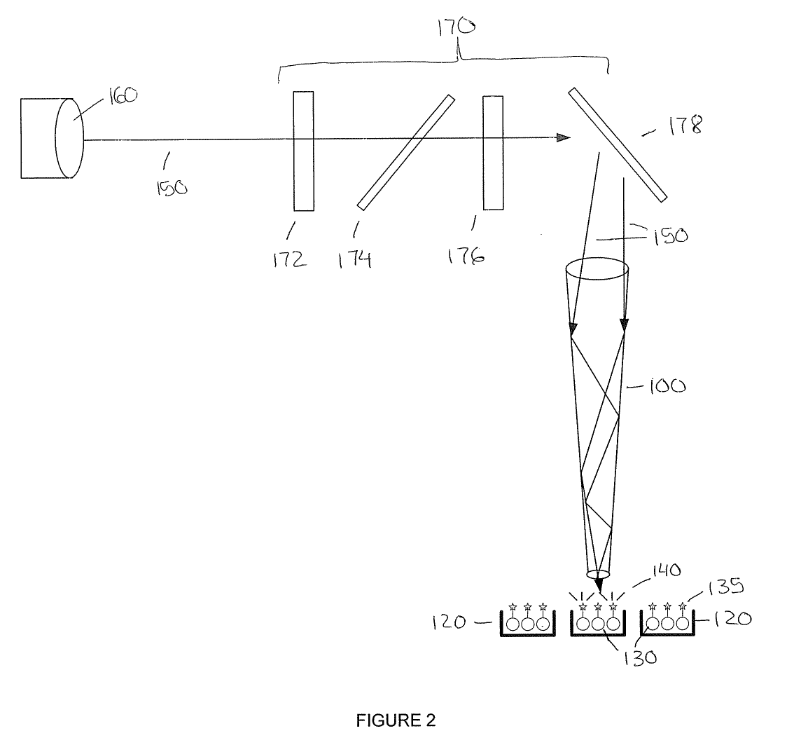

With reference to FIG. 2, a concentrator 100 is illustrated to operate in connection with the concentration of excitation light 150 emitted from a light source 160. The light source 160 may further be used in connection with various optical components 170 as previously described. In certain embodiments, the concentrator 100 may be configured to act in coordination with a focusing lens 172 to focus/condition the excitation light 150 in a desired manner so as to illuminate one or more sample containers 120. The focusing/conditioning of the excitation light 150 may further be directed at a single sample container 120 or a portion thereof to selectively illuminate the labeled sample 130 contained therein. In various embodiments, the concentrator 100 may be adapted for use without the addition of a lens 172 to achieve a comparable focusing/conditioning of the excitation light 150 thereby reducing the overall cost/complexity of the illumination system. In various embodiments, the concentrator 100 may be configured as a frustum of a hollow, excitation light reflective cone capable of concentrating light onto a smaller area than might be the case for a focusing lens alone. In various embodiments, the concentrator 100 may comprise a aluminum-walled component with the aforementioned configuration providing suitable reflective properties. Similarly, a concentrator 100 of analogous configuration/construction may be used for concentration luminescent light 140 from a sample container 120.

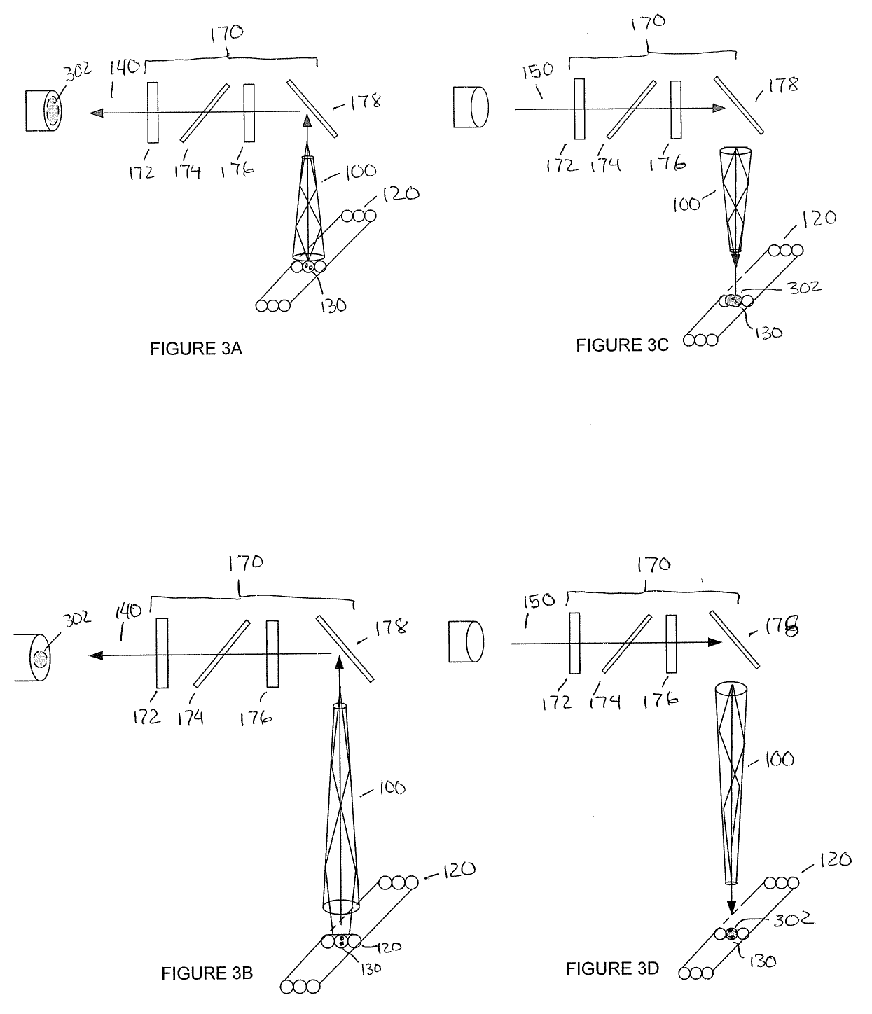

With reference to FIGS. 3A-D, a concentrator 100 may comprise a frustum of a cone or other elongate structure that concentrates the light (excitation 150 or luminescence 140) onto a larger, smaller, or equal area 302 than does a focusing lens alone. In various embodiments, greater concentrations of light may be achieved using a longer frustum (For example compare FIG. 3A to 3B and FIG. 3C to 3D). In various embodiments, the concentrator 100 may be used instead of or as a replacement to a focus lens. Using the concentrator in this context desirably eliminates the cost of the lens. As shown in FIGS. 3A-D, the concentrator 100 may be used in connection with a sample container 120 comprising a capillary. The capillary may be positioned in proximity to other capillaries and the concentrator 100 may be used to selectively illuminate and/or capture emissions from a selected sample 130. As will be appreciated by one of skill in the art, the aforementioned examples are intended as illustrative of the teachings described herein and various other configurations for the direction of excitation light 150 and the capture of luminescent light 140 may be devised including the use of separate concentrators 100 for excitation and emission optical paths as well as the use of multiple concentrators in a coordinated manner.

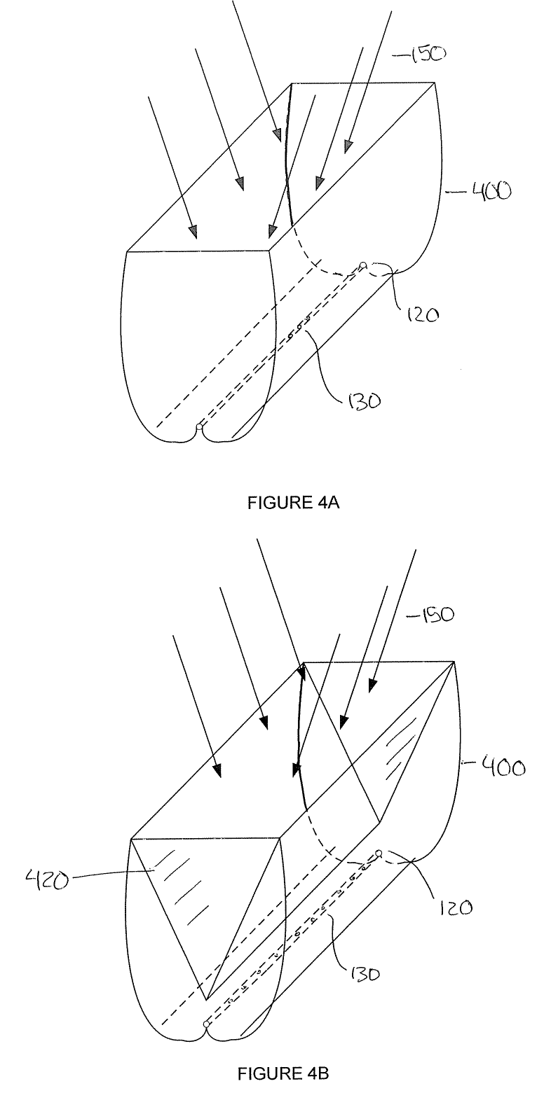

With reference to FIGS. 4A-B a concentrator may comprise a compound parabolic concentrator 400 that concentrates the light (excitation or luminescent emissions) onto a designated area. In various embodiments the concentrator 400 may be used instead of or as a replacement to a focus lens. Using the concentrator 400 in this context desirably eliminates the cost of the lens and may be used to concentrate light along an area such as a section of the sample container 120. In the illustrated embodiments, the sample container 120 comprises a capillary and the concentrator 400 is configured to illuminate a section or region of the capillary with excitation light 150. Similarly, a concentrator may be devised to concentrate luminescent emissions arising from a sample 130 retained in the sample container 120 wherein the sample 130 is distributed along a portion or region of the sample container 120 and where the concentrator 400 concentrates these luminescent emissions in a desired manner or with selected characteristics.

Additionally, as shown in FIG. 4B the concentrator may comprise a hollow or partially hollow compound hyperbolic concentrator 400 that concentrates the light as described above. As previously described, the concentrator 400 may employ the use of a reflective coating 420 for concentrating the light or alternatively a material/coating may be used in connection with the concentrator 400 that permits light 140, 150 to be propagated through the concentrator 400 using TIR. As shown in FIGS. 4A-B various embodiments of the concentrator 400 may be configured for use with an elongate sample container 120 such as a tube/capillary. The concentrator 400 may be adapted to concentrate light along a length or portion of the capillary in such a manner so as to distribute excitation light in a more uniform manner or with a higher intensity of light as compared to not using the concentrator 400. Similarly, the concentrator 400ay be configured so as to collect luminescent emissions from the elongate sample container 120 and distribute the emissions in a more uniform manner.

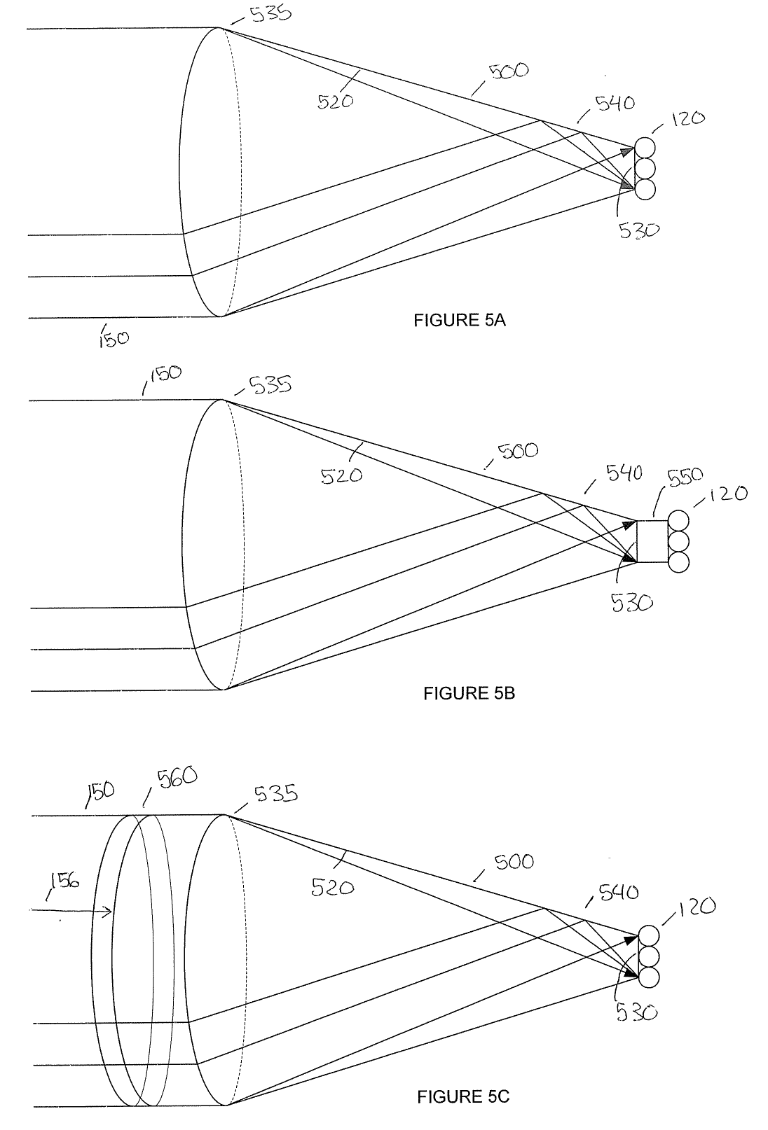

With reference to FIGS. 5A-C, an exemplary concentrator 500 having properties of TIR is shown in side view. In this exemplary configuration, the light 150 encountering the interior wall or surface portions 520 of the concentrator 500 may be adapted to converge at an exit location 530 for the concentrator 500. Light 150 encountering certain portions of the concentrator 500 can exit at an angle 535 that barely satisfies TIR (the maximum concentration method) or in parallel (the phase-conserving method). In various embodiments, such as a cone configuration, if the distance from the top edge of the concentrator to the top hyperbola focus approximately equals the distance from the top edge of the concentrator to the bottom focus, then the wall becomes substantially a straight line. In various embodiments, the light encountering the concentrator wall surface along a selected region 540 leave the concentrator 500 with approximately the same angle. Such a configuration can be useful to mapping light into a desired fraction of a sample acceptance angle. Such a configuration may be useful in illuminating a portion of a sample container 120 (e.g. capillary, well, cuvette, etc.)

With reference to FIGS. 5B-C, a coupling element comprising an exit conditioning component 550 or an entrance conditioning component 560 may be used in connection with the concentrator 500. In various embodiments, the conditioning components 550, 560 may comprise filters such as short pass filters, long pass filters, dielectrics, or diffractive gratings with selected wavelength absorbing/excluding properties. The coupling element 550, 560 may be used to exclude undesired wavelengths of light 156 from either entering or exiting the sample container 120 or alternatively may select for desired wavelengths 150 the enter or exit the sample container 120. In various embodiments, the coupling element 550, 560 may comprise a solid, semi-solid, or liquid composition selected for its index matching properties. In one such embodiment an index matching gel or hardened cement may be configured to transmit light to or from the sample container 120. Additionally the coupling element 550, 560 may comprise a compliant material such as a gel or matrix with or without index matching properties that optically couples the concentrator 500 with the sample container 120. One of skill on the art will appreciate the potential benefits of using such a coupling element 550, 560 in terms of its ability to optically couple the sample container 120 with the concentrator 500 as well as perform functions of selecting for or excluding particular wavelengths of excitation or emission light.

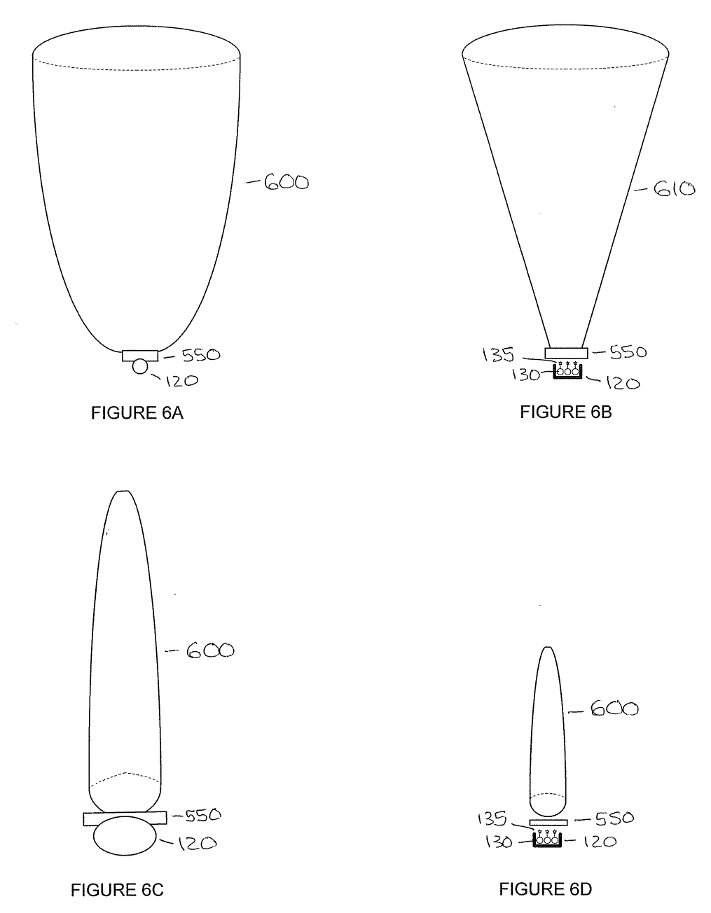

With reference to FIGS. 6A-D, representations of a dielectric compound parabolic concentrator 600 and a wedge concentrator 610 are depicted to illustrate several of many possible configurations of the concentrator. The wedge concentrator 610 may comprise a substantially straight walled cone. As with other embodiments the concentrators 600, 610 may be solid, hollow, or partially hollow and coated, doped, or fabricated in various manners to alter the optical properties of the concentrators in a desired manner. It will be appreciated that other concentrator configurations may also be used in connection with the aforementioned teachings. Additionally, excitation light 150 may selectively illuminate a particular sample container 120 or portion thereof when multiple containers are located in proximity to one another. Likewise, luminescent light 140 may be selectively collected from a particular sample container 120. Additionally, various coupling elements 550 may be used in the manners described above to moderate the characteristics of the excitation light 150 and/or luminescent light 140.

All publications and patent applications referred to herein are hereby incorporated by reference to the same extent as if each individual publication or patent application was specifically and individually indicated to be incorporated by reference.

For the purposes of this specification and appended claims, unless otherwise indicated, all numbers expressing quantities, percentages or proportions, and other numerical values used in the specification and claims, are to be understood as being modified in all instances by the term “about” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a mask” includes two or more different masks. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

What is claimed is:

1. An system for illuminating a biological sample having a responsive luminescent label, the system comprising:
an illumination source that emits energy of a type capable of a generating a response in the luminescent label;
a concentrator that receives at least a portion of the energy emitted by the illumination source and concentrates the received energy as the energy propagates through said concentrator and wherein the concentrator is configured to emit said concentrated energy with characteristic properties; and
a sample container containing the biological sample and adapted to receive the concentrated energy emitted from the concentrator wherein the concentrated energy effectuates a detectable luminescent emission arising from the label of the biological sample.

2. The illumination system of claim 1 wherein, the concentrator increases the effective illumination of the sample by increasing the relative amount of energy transmitted to the sample by the illumination source.

3. The illumination system of claim 1 wherein, the biological sample comprises one or more labeled biological molecules that are responsive to the energy emitted by the illumination source resulting in the detectable luminescent emission which is detected by a detector configured to receive the detectable emission emitted by the labeled biological molecules in the sample container.

4. The illumination system of claim 3 wherein, the biological sample comprises one more species of labeled proteins, labeled nucleic acids, or combinations thereof.

5. The illumination system of claim 1 wherein, the energy emitted by the illumination source effectuates detectable luminescent emissions in the label comprising fluorescent or phosphorescent emissions capable of being detected by the detector.

6. The illumination system of claim 1 wherein, the illumination source comprises a coherent or non-coherent source of energy.

7. The illumination system of claim 6 wherein, the illumination source comprises one or more LEDs, one or more lasers, one or more arc lamps, one or more filament lamps or any combination thereof.

8. The illumination system of claim 1 further comprising, one or more optical elements including lenses, gratings, or filters that focus, condition, or propagate the energy emitted by the illumination source.

9. The illumination system of claim 1 wherein, the characteristic properties of the energy emitted by the concentrator include an increase in the effective intensity of the energy, selected energy of a desired wavelength range, selected energy propagated in a desired area.

10. The illumination system of claim 1 wherein, the concentrator configuration is generally conical or parabolic.

11. The illumination system of claim 1 wherein, the concentrator is configured to propagate at least a portion of the energy within the concentrator by total internal reflection.

12. The illumination system of claim 1 further comprising, a conditioning filter that allows passage or exclusion of a selected wavelength range of energy.

13. The illumination system of claim 1 wherein, the sample container comprises a sample well retaining the biological sample, a capillary through which the sample passes, a channel containing the sample, a sample cuvefte, or a microcard.

14. The illumination system of claim 1 wherein, the luminescent emissions arise from a label selected from the group consisting of dye labels, fluorescent luminophores, quantum dots, and upconverting phosphors.

15. The illumination system of claim 1 wherein, the concentrator is positioned in close proximity to the sample container.

16. The illumination system of claim 15 further comprising a filter interposed between the concentrator and the sample container and having an index matching property to provide optical coupling between the concentrator and the sample container.

17. The illumination system of claim 16 wherein the index matching filter comprises an index matching fluid, an optical adhesive, a compliant optically transmissive gelatinous compound, or an index matching gel.

18. The illumination system of claim 16 wherein the filter comprises a short pass or long pass filter.

19. The illumination system of claim 16 wherein the short pass filter comprises a dielectric or absorbing filter.

20. A system for detecting luminescent emissions arising from a label associated with a biological sample, the system comprising:
a sample container containing the biological sample from which luminescent emissions arising from the label are emitted;
a concentrator that receives at least a portion of the luminescent emissions arising from the label and concentrates the luminescent emissions they are propagated through said concentrator and wherein the concentrator is configured to emit said concentrated luminescent emissions with characteristic properties; and
a detector that receives the concentrated luminescent emissions and generates a signal in response to the received concentrated luminescent emissions.

21. The detection system of claim 20 wherein, the signal generated by the detector is proportional to the relative amount of label contained in the sample container.

22. The detection system of claim 20 further comprising, an illumination source that emits energy of a type capable of a generating a response in the luminescent label in the form of the luminescent emission wherein the energy from the illumination source is directed towards the sample container.

23. The detection system of claim 20 wherein the concentrator increases the effective luminescent emission received by the detector.

24. The detection system of claim 20 wherein, the biological sample comprises one or more labeled biological molecules that are responsive to the energy emitted by the illumination source resulting in the detectable luminescent emission which is detected by a detector configured to receive the detectable emission emitted by the labeled biological molecules in the sample container.

25. The detection system of claim 20 wherein, the biological sample comprises one more species of labeled proteins, labeled nucleic acids, or combinations thereof.

26. The detection system of claim 20 wherein, luminescent emissions arising from the label comprise fluorescent, phosphorescent, or chemiluminescent emissions capable of being detected by the detector.

27. The detection system of claim 20 further comprising one or more optical elements including lenses, gratings, or filters that focus, condition, or propagate the luminescent emissions emitted by labeled biological sample.

28. The detection system of claim 20 wherein the characteristic properties of the luminescent emissions emitted by the concentrator include an increase in the effective intensity of the emissions, selected emissions of a desired wavelength range, selected emissions propagated in a desired area.

29. The detection system of claim 20 wherein the concentrator configuration is generally conical or parabolic.

30. The detection system of claim 20 wherein the concentrator is configured to propagate at least a portion of the luminescent emissions within the concentrator by total internal reflection.

31. The detection system of claim 20 further comprising a conditioning filter that allows passage or exclusion of a selected wavelength range of energy.

32. The detection system of claim 20 wherein the sample container comprises a sample well retaining the biological sample, a capillary through which the sample passes, a channel containing the sample, a channel containing the sample, a sample cuvette, or a microcard.

33. The detection system of claim 20 wherein, the luminescent emissions arise from a label selected from the group consisting of dye labels, fluorescent luminophores, quantum dots, and upconverting phosphors.

34. The detection system of claim 20 wherein, the concentrator is positioned in close proximity to the sample container.

35. The detection system of claim 20 further comprising a filter interposed between the concentrator and the sample container and having an index matching property to provide optical coupling between the concentrator and the sample container.

36. The detection system of claim 20 wherein the index matching filter comprises an index matching fluid, an optical adhesive, a compliant optically transmissive gelatinous compound, or an index matching gel.

37. The detection system of claim 20 wherein the filter comprises a short pass or long pass filter.

38. The detection system of claim 20 wherein the short pass filter comprises a dielectric or absorbing filter.
